(19) United States
(12) Patent Application Publication
(10) Pub. No.: US 2018/0143180 A1
(43) Pub. Date: May 24, 2018

(54) SYSTEM AND METHOD FOR DETERMINING TUMOR INVASIVENESS

(71) Applicant: University of Southern California, Los Angeles, CA (US)

(72) Inventors: Robert H. CHOW, Pasadena, CA (US); Jae Youn HWANG, Los Angeles, CA (US); Nan Sook LEE, Pasadena, CA (US); K. Kirk SHUNG, Monterey Park, CA (US); Andrew C. WEITZ, Pasadena, CA (US)

(21) Appl. No.: 15/616,275

(22) Filed: Jun. 7, 2017

Related U.S. Application Data
(63) Continuation of application No. 14/040,253, filed on Sep. 27, 2013, now abandoned.
(60) Provisional application No. 61/706,640, filed on Sep. 27, 2012.

(51) Int. Cl. G01N 33/50

(52) U.S. Cl. CPC G01N 33/5044; G01N 33/5017

(57) ABSTRACT

A method of determining invasion potential of a tumor cell includes exposing a tumor cell to an activity sensor; after exposing the tumor cell to the activity sensor, stimulating the tumor cell to cause a response in the cell that is reported by the activity sensor; detecting the level of response after stimulation of the tumor cell; and determining the invasion potential of the tumor cell based on the response. A system for determining the invasion potential of a tumor cell includes a sample stage that supports the tumor cell; a stimulator that focuses energy on the tumor cell to stimulate the tumor cell; and an imaging apparatus that observes an effect of the beam on the tumor cell.

### CROSS-REFERENCE TO RELATED APPLICATIONS

This application is based upon and claims priority to U.S. Provisional Patent Application No. 61/706,640, entitled “MEDICAL IMAGING APPARATUS AND METHOD FOR DETERMINING CANCER INVASIVENESS” filed Sep. 27, 2012, attorney docket number 028080-0790, which is incorporated by reference.

### STATEMENT REGARDING FEDERALLY SPONSORED RESEARCH

This invention was made with Government support under Grant Nos. R01-EB012058 and P41-EB2182, awarded by the National Institutes of Health. The Government has certain rights in the invention.

### TECHNOLOGICAL FIELD

The present disclosure relates to the field of cancer detection, and more particularly, to systems and methods of determining the degree of invasiveness of tumor cells.

### BACKGROUND

Cancer is a leading cause of death in the world. In 2013, more than 1.5 million Americans are expected to be diagnosed with cancer, and more than 500,000 will die from the disease. Breast cancer is the leading cancer in women and the second leading cause of female cancer death. Once a patient is diagnosed with a tumor, doctors must establish how aggressively to treat the patient. This requires determining whether the tumor is invasive (i.e., able to spread throughout the body), which generally requires a biopsy (removal of tissue) and subsequent laboratory analysis. One of the most devastating events for breast cancer patients is discovery of metastases, as metastasis is associated with a poor prognosis. Thus, early determination of the invasion potential of tumor cells would greatly facilitate decisions regarding the aggressiveness of therapy after cancer diagnosis.

The standard methods for determining tumor aggressiveness require a biopsy and/or genomic testing. Biopsy specimens are sent to a laboratory for analysis. The surgical removal of tissue can be painful and expensive, while the laboratory test can take several days or weeks, depending on technician availability. Biopsy specimens are sectioned, stained, and examined by a pathologist under a microscope to determine the pathological grade of the tissue. Pathological grading is based on the appearance of the tissue and requires substantial training.

Currently, the standard quantitative method to assess tumor cell invasion potential is an assay of cell penetration through a Matrigel barrier. Consequently, this method has been used to investigate the molecular mechanisms of tumor cell invasion, anticancer drug screening, development of new chemotherapy agents, and selection of invasive cellular subpopulations. Although the Matrigel invasion assay is useful for assessing the invasion potential in cells in vitro, it is not suitable for rapid determination of invasiveness of tumor cells either in vitro or ex vivo, since the method requires time-consuming establishment of cell cultures from tumor biopsies (not always successful) and then at least 24 h to complete the assay.

Therefore, the development of a new methodology that enables rapid determination of the invasiveness of tumor cells in vitro, ex vivo, and possibly in vivo would be beneficial to characterize tumor cell invasion processes, screen anticancer drugs, and, furthermore, to decide the aggressiveness of clinical therapeutic strategy.

### SUMMARY

In order to overcome the above-mentioned problems, this disclosure identifies a method of determining invasion potential of tumor cells.

In some embodiments, the method includes exposing a tumor cell to an activity sensor. After exposing the tumor cell to the activity sensor, the tumor cell is stimulated to cause a response that is reported by the activity sensor. The level of response may then be detected after stimulation of the tumor cell. The invasion potential of the tumor cell may be determined based on the response.

In some embodiments, the activity sensor comprises a fluorescent activity indicator. The fluorescent activity indicator may detect calcium ions.

In some embodiments, the stimulation includes electromagnetic and/or mechanical modalities. The stimulation may be performed by at least one of the group consisting of magnetic, electrical, ultrasound, millimeter wave, and mechanical.

In another embodiment, the stimulation is generated by a high frequency focused ultrasound transducer. In certain embodiments, the term high frequency focused ultrasound includes frequencies of from about 1 MHz to about 200 MHz, or greater.

The response may include an emission of photons or radiofrequency energy from the activity sensor. The level of response may be quantified by measuring cytoplasmic Ca²⁺ elevations induced by the stimulation of the tumor cell. The cytoplasmic Ca²⁺ elevations induced by the stimulation may be detected by a fluorescence microscope or photodetector, and may be analyzed by a computer processor. The level of response quantified by the cytoplasmic Ca²⁺ elevations may be proportional to the invasion potential of the tumor cell.

In some embodiments, the activity sensor may be carried in a solution comprising an extracellular buffer sufficient to maintain viability of the tumor cell during the step of exposing the tumor cell to the activity sensor.

The present disclosure is also directed toward a system for determining the invasion potential of a tumor cell. The system may comprise a sample stage having a configuration that supports the tumor cell, a stimulator having a configuration that focuses electromagnetic energy on the tumor cell to stimulate the tumor cell, and a microscope apparatus having a configuration that observes an effect of the stimulation on the tumor cell.

The stimulation may be at least one of the group consisting of magnetic, electrical, ultrasound, and millimeter wave. The ultrasound beam may be generated by high frequency ultrasound. In some embodiments, the ultrasound frequency may range from about 1 MHz to about 200 MHz.

The microscope apparatus may further comprise a photodetector having a configuration that receives fluorescence emissions from the tumor cell, and a light source having a configuration that provides light to the tumor cell in order to excite a fluorescent activity sensor.

In certain embodiments, the fluorescence emissions include quantification of cytoplasmic Ca²⁺ elevations induced by the stimulation of the tumor cell.

In other embodiments, the system includes a computer processor having a configuration that analyzes the cytoplasmic Ca²⁺ elevations induced by the stimulation of the tumor cell.

### BRIEF DESCRIPTION OF THE DRAWINGS

The drawings disclose illustrative embodiments. They do not set forth all embodiments. Other embodiments may be used in addition or instead. Details which may be apparent or unnecessary may be omitted to save space or for more effective illustration. Conversely, some embodiments may be practiced without all of the details which are disclosed. When the same numeral appears in different drawings, it refers to the same or like components or steps.

FIG. 1 illustrates a diagram of a system for determining the invasiveness of tumor cells according to one embodiment of the present disclosure.

FIGS. 2A-B show an image and a quantified measurement of fluorescence of a tumor cell labeled with a fluorescent calcium indicator that has been stimulated with a high frequency ultrasonic beam according to another embodiment of the present disclosure.

FIGS. 3A-B show an image of weakly invasive MCF-7 tumor cells before and during stimulation according to one embodiment of the present disclosure.

FIGS. 4A-B show an image of highly invasive MDA-MB-231 tumor cells before and during stimulation according to one embodiment of the present disclosure.

### DETAILED DESCRIPTION

Illustrative embodiments are now discussed and illustrated. Other embodiments may be used in addition or instead. Details which may be apparent or unnecessary may be omitted to save space or for a more effective presentation. Conversely, some embodiments may be practiced without all of the details which are disclosed.

The invasive nature of various malignant breast tumor cells such as MDA-MB-231 and MCF-7 has been established in many previous breast cancer studies that show that MDA-MB-231 cells are highly invasive, whereas MCF-7 cells are weakly invasive. In addition, the invasiveness in MDA-MB-231 cells can be reduced with anticancer drug treatment. These differences may be utilized to demonstrate a novel method for quickly and accurately determining the invasion potential of a tumor cell.

In some embodiments of the present disclosure, a method of determining invasion potential of a tumor cell includes exposing a tumor cell to an activity sensor. Activity sensors interact with chemical moieties and then, upon being irradiated, emit a response, such as an easily detectable signal which allows a user to quantify the level of the specific chemical moiety in the tumor cell.

In some embodiments, the activity sensor may be a fluorescent activity indicator. Fluorescent activity indicators can be loaded into cells and then viewed using a fluorescence microscope and captured by a photodetector, such as a charge-coupled device (CCD) camera. The CCD images may be analyzed by measuring fluorescence intensity changes for a single wavelength or two wavelengths expressed as a ratio (ratiometric indicators). The derived fluorescence intensities and ratios may then be plotted against calibrated values for known element or ion levels to determine the concentration.

Fluorescent activity indicators may be tailored to interact with specific elements or ions. Examples of elements or ions that may interact with a fluorescent activity indicator include zinc, copper, iron, lead, cadmium, mercury, nickel, cobalt, aluminum, lanthanides, Mg²⁺ and Ca²⁺. Fluorescent activity indicators that are specific to Ca²⁺ include, but are not limited to, fura-2, indo-1, fluo-3, fluo-4, fluo-4 AM, and Calcium Green-1. The amount of fluorescent activity indicator used should be sufficient to allow observation of the fluorescence emitted by the tumor cell.

In other embodiments, the activity sensor may be a luminescent sensor, or a magnetic resonance imaging (MRI) contrast agent. An example of a luminescent sensor sensitive to Ca²⁺ is aequorin. An example of a calcium-sensitive MRI contrast agent is DOPTA-Gd.

In other examples, the activity sensor may be a genetically encoded activity sensor. Examples of genetically encoded activity sensors include GCaMP6 or TN-XXL.

To expose the tumor cell to the activity sensor, the tumor cell may be maintained in any growth medium suitable to maintain the viability of the tumor cell during analysis. The activity sensor may then be added to, or formulated with the growth medium to contact the tumor cell.

After exposing the tumor cell to the activity sensor, the tumor cell is stimulated to cause a response from the activity sensor. In some embodiments, the stimulation is via magnetic, electrical, ultrasound, millimeter wave, or mechanical. Specific techniques include high-frequency focused ultrasound, transcutaneous magnetic stimulation, and transcutaneous direct current stimulation.

In another embodiment, the stimulation may be generated by a high frequency focused ultrasound transducer. High frequency focused ultrasound works through the generation of sound waves from transducers into a target, such as a living system.

In the some embodiments, the high frequency focused ultrasound stimulation stimulates calcium elevations in the tumor cells that have interacted with the activity sensor to generate a response. The level of response may be proportional to the amount of Ca²⁺ in the cell. Since the invasion potential may be proportional to the rise in calcium induced by stimulation, the invasion potential of the tumor cell may be determined based on the response.

The response may include an emission of photons or radiofrequency energy from the activity sensor. The level of response may be quantified by measuring cytoplasmic Ca²⁺ elevations induced by the stimulation of the tumor cell. The fluorescent indicator may be generally used with the chelator carboxyl groups masked as acetoxymethyl esters, in order to render the molecule lipophilic and to allow easy entrance into the tumor cell. Once the indicator is in the tumor cell, cellular esterases will free the carboxyl and the indicator will be able to bind calcium. Binding of a Ca²⁺ ion to a fluorescent indicator molecule leads to either an increase in quantum yield of fluorescence or emission/excitation wavelength shift.

The cytoplasmic Ca²⁺ elevations induced by the stimulation may be detected by a fluorescence microscope or photodetector. The level of response quantified by the cytoplasmic Ca²⁺ elevations may be proportional to the invasion potential of the tumor cell.

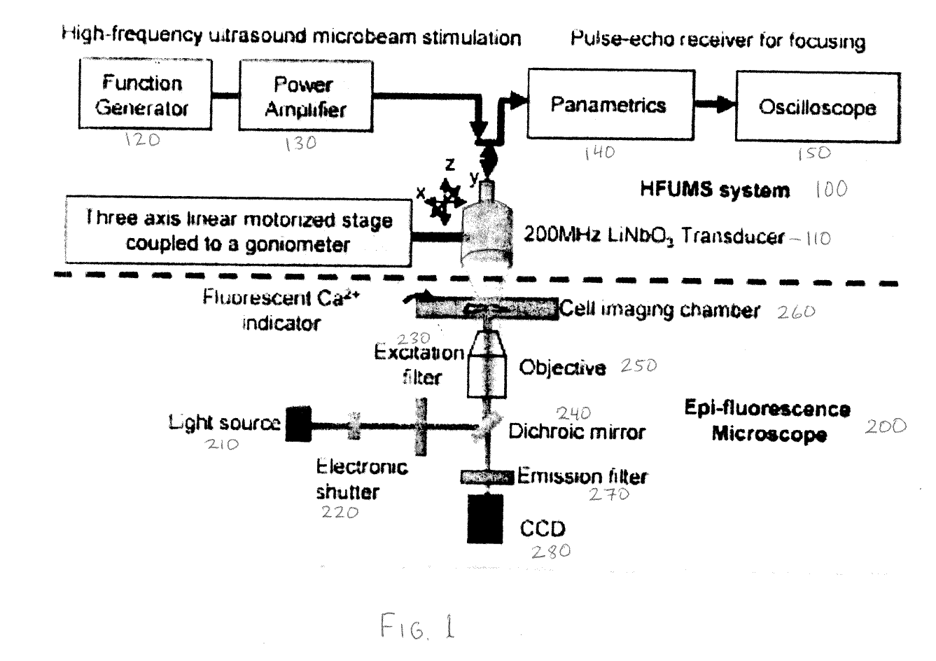

FIG. 1 shows one embodiment of a system for determining the invasion potential of a tumor cell. The system layout may include a High Frequency Focused Ultrasound (HFFU) device 100 and fluorescence imaging attachments. In the HFFU device 100, a transducer 110 may be used to generate a focused ultrasound beam for single- or multi-cell stimulation. In some embodiments, a 200-MHz transducer may be used. In other embodiments, a 35-MHz transducer may be used. A transducer of from about 1 MHz to about 200 MHz or greater may be used. The transducer may be constructed with conventional transducer fabrication procedures. In order to generate the ultrasound beam, sinusoidal bursts of the desired frequency may be emitted from a function generator 120. The function generator 120 may be fed into a power amplifier 130 to drive the transducer 110. Panametrics 140 and an oscilloscope 150 may be utilized for directing the beam to the tumor cell.

Live-cell fluorescence imaging may be carried out on a fluorescence microscope 200 to monitor the cytoplasmic Ca²⁺ elevations elicited by HFFU. A light source 210, such as a mercury lamp, may deliver light to the cells in the cell imaging chamber 260 for excitation of the activity sensor. The light may pass through an electronic shutter 220, an excitation bandpass filter 230, a dichroic mirror 240, and an objective 250 before being delivered to the cells. Fluorescence emitted from the cells may then be collected by the objective 250 and recorded using a high-sensitivity CCD camera 280 after passing through an emission bandpass filter 270.

In the CCD camera, pixels are represented by p-doped MOS capacitors.

These capacitors are biased above the threshold for inversion when image acquisition begins, allowing the conversion of incoming photons into electron charges at the semiconductor-oxide interface. The CCD is then used to read out these charges. As a result, the CCD camera is useful for scientific applications where high-quality image data is required.

In certain embodiments, the fluorescence emissions include quantification of cytoplasmic Ca²⁺ elevations induced by the stimulation of the tumor cell.

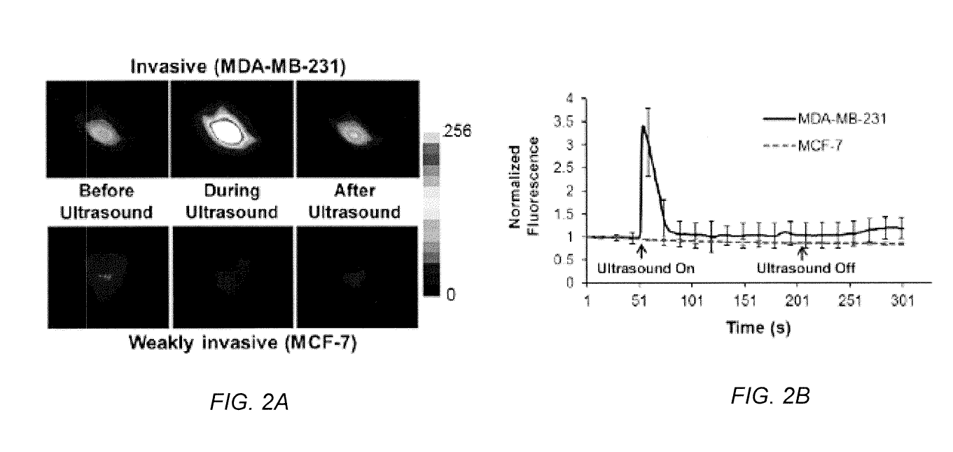

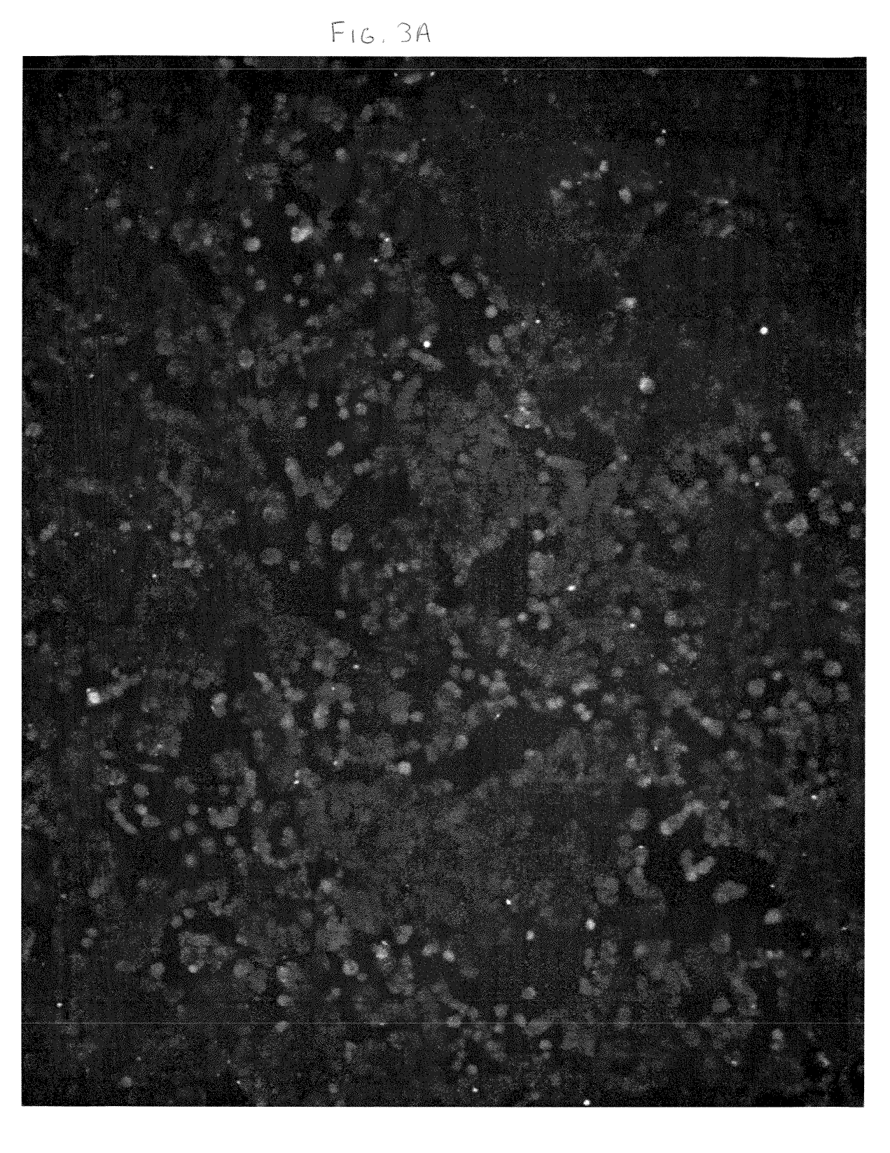

FIGS. 2A-B illustrate that HFFU elicited cytosolic calcium elevations in MDA-MB-231 cells, but not markedly in MCF-7 cells. The initiating times, durations, amplitudes, and number of transient Ca²⁺ elevations elicited by HFFU may differ slightly for individual tumor cells.

HFFU elicits cytosolic calcium elevations in highly invasive breast cancer tumor cells to a significantly greater extent than it does in weakly invasive breast cancer tumor cells. Furthermore, other methods may be used in conjunction with HFFU. HFFU can be complementarily combined with other imaging modalities such as acoustic radiation force impulse imaging, which enables the estimation of elastic properties of tumor cells in situ and in vivo. Notably, the elastic properties of tumor cells have been importantly considered as one of primary indicators in the determination of metastatic potential of tumor cells. Thus, combining measurements of HFFU-induced calcium elevation and estimation of their elastic properties may offer more accurate determination of the metastatic potential of breast tumor cells both in situ and in vivo.

Another embodiment that utilizes ex vivo procedures for determining tumor invasiveness involves a device to automatically measure invasiveness following biopsy. The surgeon could take the biopsied tissue and load it with an activity sensor, which could possibly be done by the device. The tissue could then be placed in the device for analysis. While maintaining tissue health, the device would stimulate the tumor with an electromagnetic and/or mechanical modality, and use a photodetector, such as a CCD sensor, to capture photons emitted by the activity sensor. A computer processor could then be used to measure/determine the invasive potential, and report/display the invasion potential to the physician. The device may also include electronics for driving the stimulator. For example, in the case of ultrasound stimulation, hardware may be used to generate a sine wave.

In other embodiments, in vivo procedures could be used. For example, a tumor could be labeled with an activity sensor, either by injecting the sensor into the bloodstream or using a needle or catheter to deliver the sensor directly to the tumor. A catheter could be positioned near the tumor. In one example, the catheter could include three individual lumens, or tubes, combined in a single housing and mechanically isolated from one another. The first lumen may be an infusion lumen used for delivering the activity sensor to the tumor. The infusion lumen is not required if the sensor is delivered through the bloodstream. The second lumen may be used to stimulate the tumor, such as with an ultrasound transducer. The third lumen may be a fiber optic lumen for providing light to excite the fluorescent sensor and for capturing the photons the fluorescent sensor emits. Emitted photon flux may be used to determine the degree of invasiveness.

In another in vivo example, the tumor could be labeled with a red-shifted activity sensor, which may emit a wavelength of light that can pass through the body. Labeling can be accomplished by injecting the sensor into the bloodstream or using a needle or catheter to deliver the sensor directly to the tumor. The tumor could be stimulated noninvasively. For example, stimulation could pass through the patient's body and be focused on the tumor. Photons emitted from the sensor could travel through the body and be captured by an external photodetector. Emitted photon flux may be used to determine the degree of invasiveness.

In yet another in vivo example, a tumor could be labeled with a calcium-sensitive MRI contrast agent. Labeling can be accomplished by injecting the agent into the bloodstream or using a needle or catheter to deliver the sensor directly to the tumor. The patient could be placed in an MRI machine. The tumor may be stimulated noninvasively, and the signal from the MRI agent could be used to determine the degree of invasiveness.

In an embodiment that utilizes mechanical stimulation, the cells could be poked with a needle or pipette, or placed in a hypo-osmotic solution to stretch the cell membrane.

Examples of the present disclosure are shown and described herein. It is to be understood that the disclosure is capable of use in various other combinations and environments and is capable of changes or modifications within the scope of the inventive concepts as expressed herein.

### EXAMPLES

Pre-Cell Preparation and Materials

MDA-MB-231, MCF-7, SKBR3, and BT-474 human breast cancer cell lines were obtained from the ATCC, and maintained in a modified complete medium (RPMI, 10% fetal bovine serum, 10 mM HEPES, 2 mM L-glutamine, 1 mM sodium-pyruvate, 0.05 mM 2-mercaptoethanol, 11 mM D-glucose). A calcium indicator, Fluo-4 AM, was purchased from Invitrogen (Grand Island, N.Y.) for live-cell calcium fluorescence imaging. Taxol was obtained from Sigma-Aldrich (Saint Louis, Mo.). During HFFU stimulation, cells were maintained in an extracellular buffer containing (in mM): 140 NaCl, 2.8 KCl, 10 HEPES (titrated to pH 7.4 with NaOH), and 1 MgCl₂.6H₂O, 2 CaCl₂.2H₂O, and 10 D(+) glucose.

HFFU Stimulation and Live Cell Calcium Fluorescence Imaging System

In order to perform live-cell fluorescence imaging of target cells stimulated by HFFU, a HFFU stimulation system was added to an inverted epifluorescence microscope (Olympus IX70). In order to generate the highly focused ultrasound beam for single cell stimulation, a 200-MHz press-focused LiNbO₃ transducer (fc: 200 MHz and bandwidth: 29%) was used. The transducer was constructed with conventional transducer fabrication procedures (Lam et al., 2013). The focal length of the transducer was 1.3 mm and the f-number (F#) was 1.6. The measured beam width of the highly focused ultrasound beam was 17 μm, which was close to the predicted value of 12 μm (=focal length×wavelength) and approximately the size of a breast cancer cell. In order to generate the 200-MHz ultrasound beam, 200-MHz sinusoidal bursts from a function generator (Stanford Research Systems, Sunnyvale, Calif.) fed into a 50-dB power amplifier (525LA, ENI, USA) were used to drive the transducer. The resultant peak-to-peak (Vₚₚ) voltages of the bursts were adjusted to 4, 8, 16, and 32 V. The duty factor was tuned to 1%, and the pulse repetition frequency (PRF) was 1 kHz.

Live-cell fluorescence imaging was carried out on the epifluorescence microscope to monitor the cytoplasmic Ca²⁺ elevations elicited by HFFU in individual MDA-MB-231, MCF-7, SKBR3, and BT-474 cells labeled with Fluo-4 AM. Light from a mercury lamp was delivered to the cells for excitation after passing through an electronic shutter, an excitation bandpass filter (488±20 nm), a dichroic mirror (cut-off wavelength: 500 nm), and a 20×objective. Fluorescence emitted from the cells was then collected by the same objective and recorded using a high-sensitivity CCD camera (ORCA-Flash2.8, Hamamatsu, Japan) after passing through an emission bandpass filter (530±20 nm).

Live-Cell Calcium Fluorescence Imaging

Fluo-4 AM was used for live-cell calcium fluorescence imaging. 10⁵ cells were plated on 35 mm Petri dishes and incubated in the complete medium at 37° C. for 36 h before 1 μM Fluo-4 AM solution, diluted with the external buffer solution, was added to the dishes. After the cells were incubated at room temperature for 30 min, the cells were thoroughly washed with external buffer solution and then time-lapse fluorescence imaging was initiated after the target cells were positioned at the beam focus. Fluorescence images were acquired at 1 Hz for t=300 s (exposure time: 300 ms), as the HFFU was switched on and off at t=50 s and t=200 s, respectively.

Quantitative Analysis for Cytoplasmic Ca²⁺ Elevations in Individual Cells

Quantitative analysis of Ca²⁺ changes in MDA-MB-231, MCF-7, SKBR3, and BT-474 cells was achieved with in-house software. The program was written to obtain the mean normalized maximum calcium elevation value and a cell responding ratio from segmented images of target cells, semi-automatically. A cell responding ratio=the number of cells responding to HFFU/the number of total cells subjected to HFFU. More specifically, after fluorescence images of cells acquired at different time-points (0-300 s, step: 1 s) were averaged, the target cell receiving HFFU in the averaged image was selected and segmented by Otsu's method (Otsu, 1979), followed by the calculation of mean fluorescence intensities in the segmented regions of each image obtained at the indicated time-points. Temporal changes of the mean fluorescence in the target cell were then analyzed to determine whether calcium elevations were elicited by HFFU in the cell. The calcium elevation was here measured as the increase in the fluorescence intensity. In the cell exhibiting calcium elevations, the maximum calcium elevation value with HFFU stimulation of the cells was normalized to the mean value of fluorescence intensities (control) obtained prior to HFFU (Ozkucur et al., 2009).

Finally, after the normalized maximum calcium elevations obtained from independent cells (n>9) were averaged, the mean value was multiplied by the cell responding ratio to give a composite parameter, called the cell response index (CRI), where a larger CRI indicates a stronger response to HFFU. Use of the cell responding ratio in addition to magnitude of Ca²⁺ elevations has also been considered in other studies to quantify cellular responses to external stimuli (Bunn et al., 1990).

Effect of Ultrasound Beam Exposure Levels on Cytoplasmic Ca²⁺ Elevation in MDA-MB-231 Cells

Table 1 shows the mean and standard deviation of mean maximum Ca²⁺ elevation×cell responding ratio at variable input voltages to the transducer in MDA-MB-231 (n=9).

The degree of responsiveness among MDA-MD-231 cells based on the amplitude of the voltage driving the HFFU transducer was analyzed. As is shown in Table 1, when the voltage inputs were 4 V and 8 V, the CRI values significantly increased up to almost a fourfold increase over the baseline value (0 V input; P-value: 6.7×10⁻⁷). Also, the CRI values increased more as the input voltages were increased. These results demonstrate that there is a dose-response relationship between the CRI value and acoustic pressure.

TABLE 1

Input Voltage (Vₚₚ)

| | 0 V | 4 V | 8 V | 16 V | 32 V |
|---|---|---|---|---|---|
| Mean | 0.33 | 1.31 | 1.25 | 1.76 | 2.08 |
| Standard | 0.14 | 0.26 | 0.20 | 0.37 | 0.68 |
| Deviation | | | | | |

Taxol Treatment

Taxol is known to inhibit tumor growth as well as reduce the invasiveness of tumor cells. HFFU stimulation of cells treated with a range of Taxol concentrations was performed. In order to investigate the effects of the anticancer agent Taxol on HFFU-induced Ca²⁺ elevations in MDA-MB-231 cells, 10⁵ cells were plated in 35 mm Petri dishes and incubated in the RPMI complete medium at 37° C. for 24 h, followed by Taxol treatment of the cells at the indicated concentrations (0, 1, 10, and 100 nM). After 24 h, the cells were thoroughly washed with external buffer solution. Live-cell calcium fluorescence imaging of the cells (n=10) was performed during HFFU stimulation, as already described.

The normalized CRIs were 1.0 at 0 nM, 0.52 at 1 nM, 0.29 at 10 nM, and 0 at 100 nM, respectively. The results show that CRI decreases as the Taxol concentration increases. Only 1 nM Taxol was sufficient to reduce the CRI by ˜50% relative to the untreated cells. Furthermore, 100 nM Taxol reduced the CRI to 0%. Thus, the HFFU-induced Ca²⁺ elevations in MDA-MB-231 cells are correlated with their invasiveness and raise the possibility that monitoring HFFU-induced Ca²⁺ elevations in breast cancer cells may be utilized to quantify the invasiveness in the cells.

Cell Invasion Assay

Cell invasion assays were performed on 8 μm diameter pore BD BioCoat Matrigel Invasion Chambers (BD Biosciences, San Jose, Calif.) according to the manufacturer's instructions. Cells (1.5×10⁵) were added to chambers and incubated for 2 days at 37° C. Matrigel and noninvasive cells inside the chamber were removed by Q-tips, and the invasive cells that had passed through the Matrigel of the chamber were stained with 0.2% crystal violet in 10% ethanol. Absorbance (at 590 nm) of each well was measured and quantified using a plate reader (SpectraMax M2, Molecular Devices, Sunnyvale, Calif.).

Statistical Analysis

The CRIs of MDA-MB-231, MCF-7, SKBR3, and BT-474 cells were compared. All data were expressed as mean±standard deviation of indicated sample sizes, and were analyzed by a two-tailed paired t-test, with the level of significance set at P-value <0.01. The number of invading cells was quantitated from triplicate experiments.

Results

Cytoplasmic Ca²⁺ Variations in MDA-MB-231 and MCF-7 Cells Elicited by HFFU

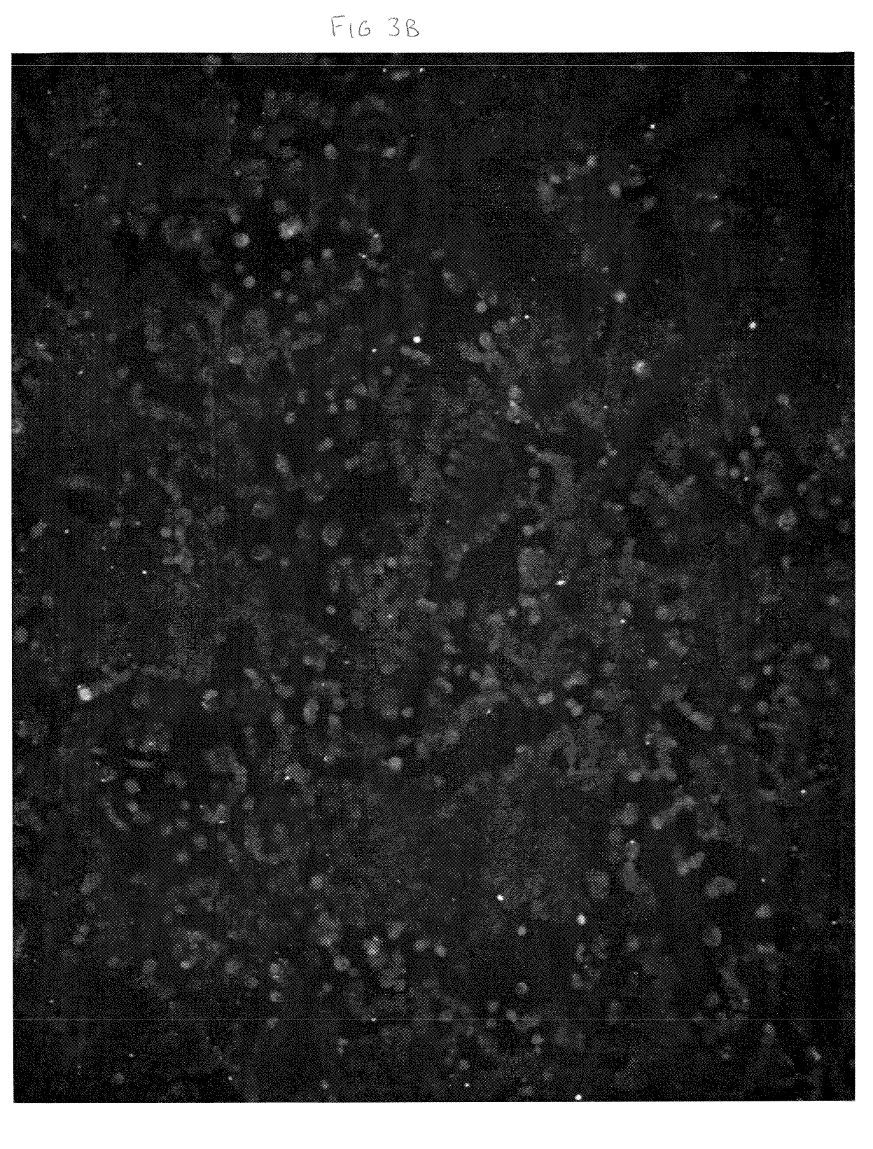

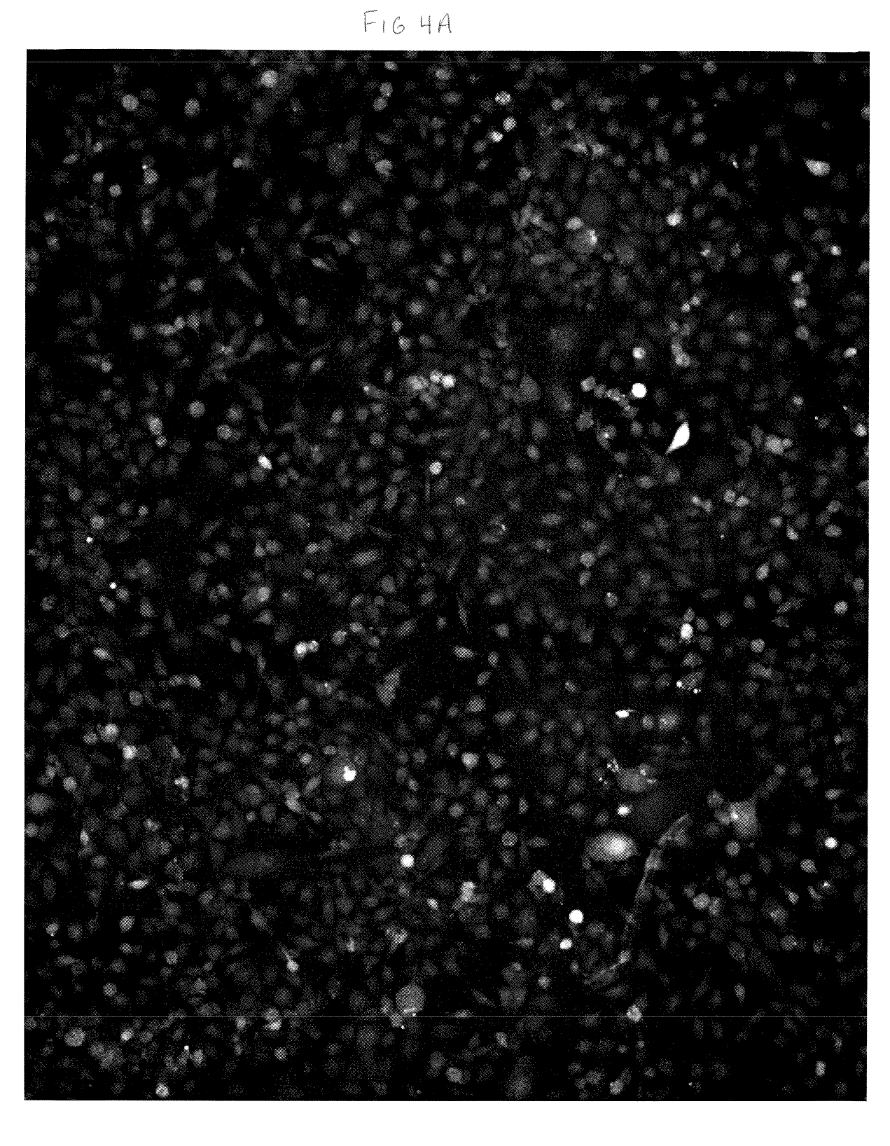

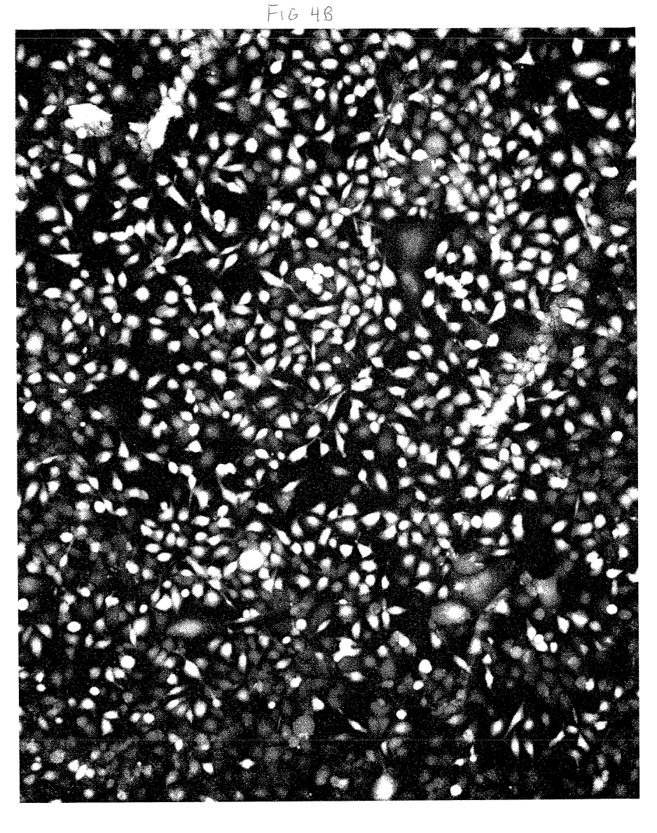

Live-cell fluorescence imaging was used to monitor Ca²⁺ changes in MDA-MB-231 (highly invasive) and MCF-7 (weakly invasive) cells, preincubated with Fluo-4 AM. HFFU elicited little to no fluorescence changes in most MCF-7 cells, as is shown in FIG. 2A lower frames. FIGS. 3A-B show the MCF-7 cells before and during HFFU stimulation with 35 MHz ultrasound. Significant fluorescence increases were observed in MDA-MB-231 cells, as is shown in FIG. 2A upper frames. FIG. 2B illustrates the normalized Ca²⁺ temporal variations in MDA-MB-231 and MCF-7 cells due to HFFU. FIGS. 4A-B show the MDA-MB-231 cells before and during HFFU stimulation with 35 MHz ultrasound. The MDA-MB-231 cells clearly exhibited transient Ca²⁺ elevations when HFFU was on. In contrast, in most MCF-7 cells such transient calcium elevations by HFFU were not observed. While the ultrasound beam is focused only on one or a few cells (depending on the frequency), a very large field of cells is excited. This is due to paracrine signaling (cell-to-cell communication) among the population. This can be described as a calcium wave response.

CRI Values in Breast Cancer Cells Elicited by HFFU

Ca²⁺ elevations in MDA-MB-231, MCF-7, SKBR3, and BT-474 cells subjected to HFFU were quantitated using the program described above. CRI for MDA-MB-231 cells (n=58) is significantly higher than that for MCF-7 (n=58), SKBR3 (n=40), and BT-474 (n=40) cells (P-value <0.01). The cell responding ratio of MDA-MB-231 cells was. 0.82, whereas the cell responding ratios of MCF-7, SKBR3, and BT-474 cells were. 0.24, 0.34, and. 0.26, respectively. The invasiveness in MDA-MB-231, MCF-7, SKBR3, and BT-474 cells was assessed using a Matrigel invasion chamber. Indeed, the number of MDA-MB-231 cells that passed through the Matrigel barrier was much higher than that of the other cell types. Together, these results demonstrate that the HFFU-induced Ca²⁺ elevations in MDA-MB-231 cells are significantly higher than those in MCF-7, SKBR3, and BT-474 cells, and they suggest that HFFU-stimulated calcium elevation may be used to distinguish MDA-MB-231 cells from MCF-7, SKBR3, and BT-474 cells, and perhaps may be used to determine the invasiveness of breast cancer cells.

The components, steps, features, objects, benefits and advantages which have been discussed are merely illustrative. None of them, nor the discussions relating to them, are intended to limit the scope of protection in any way. Numerous other embodiments are also contemplated. These include embodiments which have fewer, additional, and/or different components, steps, features, objects, benefits and advantages. These also include embodiments in which the components and/or steps are arranged and/or ordered differently.

Unless otherwise stated, all measurements, values, ratings, positions, magnitudes, sizes, and other specifications which are set forth in this specification are approximate, not exact. They are intended to have a reasonable range which is consistent with the functions to which they relate and with what is customary in the art to which they pertain.

All articles, patents, patent applications, and other publications which have been cited are hereby incorporated herein by reference.

What is claimed is:

1. A method of determining invasion potential of a tumor cell, comprising
exposing a tumor cell to an activity sensor;
after exposing the tumor cell to the activity sensor, stimulating the tumor cell with at least one selected from the group of an electromagnetic and mechanical modality;
detecting a level of response after stimulation of the tumor cell with an electronic sensor; and
determining the invasion potential of the tumor cell based on the response.

2. The method of claim 1, wherein the activity sensor comprises a fluorescent activity indicator.

3. The method of claim 2, wherein the fluorescent activity indicator detects calcium ions.

4. The method of claim 1, wherein the tumor cell is a cancer cell.

5. The method of claim 4, wherein the stimulation is performed by at least one of the group consisting of magnetic, electrical, ultrasound, millimeter wave, and mechanical.

6. The method of claim 5, wherein the ultrasound is generated by a high frequency focused ultrasound transducer.

7. The method of claim 1, wherein the response includes an emission of photons or radiofrequency energy from the activity sensor.

8. The method of claim 1, wherein the activity sensor is carried in a solution comprising an extracellular buffer sufficient to maintain viability of the tumor cell during the step of exposing the tumor cell to the activity sensor.

9. The method of claim 3, wherein the level of response is quantified by measuring cytoplasmic Ca²⁺ elevations induced by the stimulation of the tumor cell.

10. The method of claim 9, wherein the cytoplasmic Ca²⁺ elevations induced by the stimulation are detected by a fluorescence microscope or photodetector.

11. The method of claim 10, wherein the cytoplasmic Ca²⁺ elevations induced by the stimulation are analyzed by a computer processor.

12. The method of claim 11, wherein the level of response quantified by the cytoplasmic Ca²⁺ elevations are proportional to the invasion potential of the tumor cell.

13. The method of claim 1, wherein the tumor cell is a breast cancer cell.

14. A system for determining the invasion potential of a tumor cell, the system comprising:
a sample stage having a configuration that supports the tumor cell;
a stimulator having a configuration that focuses electromagnetic energy on the tumor cell to stimulate the tumor cell; and
an imaging apparatus having a configuration that observes an effect of the electromagnetic energy on the tumor cell.

15. The system of claim 14, wherein the electromagnetic energy is at least one of the group consisting of magnetic, electrical, millimeter wave, and ultrasound.

16. The system of claim 15, wherein the ultrasound is generated by a high frequency focused ultrasound transducer.

17. The system of claim 16, wherein the high frequency focused ultrasound transducer is tuned to a frequency of from about 1 MHz to about 200 MHz.

18. The system of claim 14, wherein the imaging apparatus further comprises:
a photodetector having a configuration that receives fluorescence emissions from the activity sensor in the tumor cell; and
a light source having a configuration that provides light to excite the activity sensor in the tumor cell.

19. The system of claim 18, wherein the fluorescence emissions include quantification of cytoplasmic Ca²⁺ elevations induced by the stimulation of the tumor cell.

20. The system of claim 14, further including a computer processor having a configuration that analyzes the cytoplasmic Ca²⁺ elevations induced by the stimulation.
